(19) United States
(12) Patent Application Publication
(10) Pub. No.: US 2006/0199244 A1
(43) Pub. Date: Sep. 7, 2006

(54) Production of open-chained sophorolipids

(76) Inventors: Richard D. Ashby, Glenside, PA (US); Daniel K.Y. Solaiman, Dresher, PA (US); Thomas A. Foglia, Lafayette Hill, PA (US); Michael J. Haas, Oreland, PA (US)

Correspondence Address: USDA, ARS, OTT, 5601 SUNNYSIDE AVE, RM 4-1159, BELTSVILLE, MD, 20705-5131, US

(21) Appl. No.: 11/324,516

(22) Filed: Jan. 3, 2006

Related U.S. Application Data
(60) Provisional application No. 60/657,453, filed on Mar. 1, 2005.

(51) Int. Cl. C12P 33/00; C12P 7/64; C12N 1/18

(52) U.S. Cl. 435/52; 435/134; 435/254.22

(57) ABSTRACT

Methods for the production of open-chained sophorolipids, involving cultivating a yeast capable of producing open-chained sophorolipids in a culture medium containing fatty acid alkyl esters, and glycerol or carbohydrates (e.g., glucose) or mixtures thereof; wherein at least about 60% of the sophorolipids produced are open-chained sophorolipids.

### REFERENCE TO RELATED APPLICATION

This application claims the benefit of U.S. Provisional Application No. 60/657,453, filed 1 Mar. 2005, which is incorporated herein by reference in its entirety.

### BACKGROUND OF THE INVENTION

The present invention relates to methods for the production of open-chained sophorolipids, involving cultivating a yeast capable of producing open-chained sophorolipids in a culture medium containing (1) fatty acid alkyl esters and (2) glycerol or carbohydrates or mixtures thereof; wherein at least about 60% of the sophorolipids produced are in the open-chained form.

As the world's energy producers are focusing more towards sustainability, corn and oilseed crops are being examined as potential starting materials to produce cheap, renewable fuels. Presently, bioethanol and biodiesel are products of intense interest to the biofuels industry. Bioethanol is typically produced using raw materials derived from starch plants (mostly corn), sugar plants (e.g., sugar beets, sugar cane), and more recently from lignocellulosic material through microbial fermentation (Dien, B. S., et al., Appl. Biochem. Biotechnol., 115:937-949 (2004); Zaldivar, J., et al., Appl. Microbiol. Biotechnol., 56:17-34 (2001); Dien, B. S., et al., Appl. Microbiol. Biotechnol., 63:258-266 (2003); Schell, D. J., et al., Bioresour. Technol., 91:179-188 (2004); Kim, S, and B. E. Dale, Biomass Bioenergy 26:361-375 (2004)). In contrast, biodiesel (i.e., methyl or ethyl esters from animal fats and/or vegetable oils) is synthesized primarily by the chemical transesterification of triacylglycerols (van Gerpen, J, and G. Knothe, in The Biodiesel Handbook, G. Knothe, J. Krahl, and J. van Gerpen, eds., AOCS Press, Champaign Ill., 2005, pp. 26-41).

Recent figures indicate that the United States produced an average of 5.5 million metric tons of animal fat and grease (47% of which is tallow) and 10.8 million metric tons of vegetable oil (77% soybean oil) annually between the years 1999 and 2001. These production numbers continue to fluctuate slightly from year to year. However, recent trends indicate a net increase in both animal fat and vegetable oil production, making it increasingly important to provide additional outlets for these commodity materials. Between the years 1998 and 2002, world production of biodiesel increased from 0.2 to 32 million gallons and, because of its fuel properties and improved emission characteristics, production is projected to reach 350 million gallons by the year 2011 (Wilson, E. K., Chem. Engineer. News, 80(21): 46-49 (2002)). Such increases in biodiesel production, while providing an additional outlet for fats and oils, will result in a large co-product stream, the disposal of which must be addressed to make such large production capacities economically and environmentally feasible. The biodiesel co-product stream (BCS) is composed primarily of glycerol, acylglycerols, free fatty acids (FFA), and residual fatty acid alkyl esters (e.g., fatty acid methyl esters (FAME) left over after the removal of the bulk of the fatty acid methyl esters in the production of biodiesel); thus the ratio of these components (i.e., glycerol, acylglycerols, free fatty acids, fatty acid alkyl esters) results from the transesterification process used to manufacture the alkyl esters and the recovery efficiency of the fatty acid alkyl esters which make up biodiesel. Because of this expected increase in the biodiesel market, it would be beneficial if BCS could be used as a feedstock for the production of additional value-added products. The possibility exists that the glycerol could be recovered, purified and sold; however, this practice is expensive, time-consuming, and would result in a glut in the glycerol market.

Sophorolipids (SLs) are extracellular glycolipids produced by yeasts (e.g., Candida bombicola) and are composed of a disaccharide (sophorose; 2-O-β-D-glucopyranosyl-β-D glucopyranose) attached to a hydroxy fatty acyl moiety at the ω-1 or ω carbon (FIG. 1) (Asmer, H-J., et al., J. Am, Oil Chem. Soc., 65:1460-1466 (1988); Hommel, R. K., and K. Huse, Biotechnol. Lett., 15: 853-858 (1993)). Typically, the 6′ and 6″ hydroxyl groups of the sophorose sugar are acetylated and the fatty acid chain length varies between 16 and 18 carbons and may be saturated or unsaturated (although recently it has been confirmed that Rhodotorula bogoriensis can synthesize SL containing fatty acid side-chains of 22 and 24 carbons (Nuñez, A., et al., Biotechnol. Lett., 26: 1087-1093 (2004)). In addition, the carboxylic acid group of the fatty acid may be lactonized (the form generally produced under typical reaction conditions) (see FIG. 1A) to the disaccharide ring at carbon 4″ or remain as the free acid in the open-chain form (see FIG. 1B).

The amphiphilic nature of SLs imparts surfactant-type properties to them and has allowed their utilization as additives in shampoo, body washes, and detergents (U.S. Pat. No. 5,417,879; U.S. Pat. No. 4,215,213), in cosmetic products (European Patent EP 0209783), and in the lubricant industry. In addition, their unique structure has increased interest in their use as a source of specialty chemicals such as sophorose and hydroxylated fatty acids (Rau, U., et al., Ind. Crops Prod., 13: 85-92 (2001)). These compounds are nontoxic, biodegradable, and are produced in large quantities by yeast (e.g., C. bombicola), thus making them an attractive target for the feedstock utilization of glycerol and BCS. Modification of the fatty acid portion of the SL structure can alter their physical and chemical properties, such as critical micelle concentration (CMC), surface active properties, and detergent properties. To a certain extent, structural variation can be achieved by changing the lipidic carbon source, which alters the SL fatty acid content (Nuñez, A., et al., Chromatographia, 53: 673-677 (2001); Davila, A-M., et al., J. Ind. Microbiol., 13: 249-257 (1994)) and may influence the acetylation pattern of the SLs (Davila, A-M., et al.; Davila, A-M., et al., Appl. Microbiol. Biotechnol., 38: 6-11 (1992); Zhou, Q. H., et al., J. Am. Oil Chem. Soc., 69: 89-91 (1992)). Recent studies showed that SLs can also be customized through the use of chemo-enzymatic reactions to produce molecules such as glucose lipids (Rau, U., et al.) and SLs with fatty acid chains of varying functionality (Bisht, K. S., et al., J. Org. Chem., 64: 780-789 (1999); Nuñez, A., et al., Biotechnol. Lett., 25: 1291-1297 (2003)). Because SLs are naturally synthesized with a preference for the lactonic form, the lactone ring must be opened prior to introducing a chemical modification at the carboxylic group of the fatty acid. While this procedure is not difficult, it does cause the deacetylation of the sophorose sugar and imparts an additional cost to the alteration of the fatty acid side-chain.

We have discovered that increased yields of the open-chain form of SLs can be produced by yeast (e.g., C. bombicola) without the need to chemically open the lactone ring prior to fatty acid modification.

### SUMMARY OF THE INVENTION

In accordance with the present invention, there is provided a method for the production of open-chained sophorolipids, involving cultivating a yeast capable of producing open-chained sophorolipids in a culture medium containing fatty acid alkyl esters, and glycerol or carbohydrates or mixtures thereof; wherein at least about 60% of the sophorolipids produced are open-chained sophorolipids.

### BRIEF DESCRIPTION OF THE DRAWINGS

FIG. 1 shows structures of 17-L-[2′-O-β-glucopyranosyl-β-D-glucopyranosyl)-oxy]-9-octadecenoic acid 6′,6″-diacetate sophorolipids in the (A) 1′,4″-lactone and (B) free acid forms.

FIG. 2 shows LC/APCI-MS (High Performance Liquid Chromatography associated with Atmospheric Pressure Chemical Ionization-Mass Spectrometry) analysis of the sophorolipid (SL) produced by Candida bombicola when grown on BCS: (A) total current ion chromatogram, (B) resulting chromatogram showing the non-lactonized products after plotting the ion mass of 409, and (C) chromatogram of the elution of the lactone SL forms after plotting the ions corresponding to the mass range of 600-700 amu.

FIG. 3 shows APCI spectrum of the peak eluting at approximately 16 minutes in FIG. 2A (R=either an acetyl group or proton).

FIG. 4 shows LC/APCI-MS analysis of the SL produced by C. bombicola when grown on pure glycerol showing the formation of lactonized SLs as the main products.

FIG. 5 shows total ion chromatograms (TICs) of the sophorolipids produced by Candida bombicola from mixed substrate fermentations of glycerol and (A) methyl soyate, (B) ethyl soyate, and (C) propyl soyate.

FIG. 6 shows positive APCI-MS of C_{18:2} diacetylated open-chain sophorolipids in the (A) free-acid (elution time≈5.2 min, see FIG. 5A) and (B) methyl ester (elution time≈13 min, see FIG. 5A) forms.

FIG. 7 shows positive APCI-MS of C_{18:2} diacetylated 1′,4″ lactonic sophorolipid (elution time≈17 min, see FIG. 5B). Note: 204 amu correspond to the loss of an acetylated hexose ring.

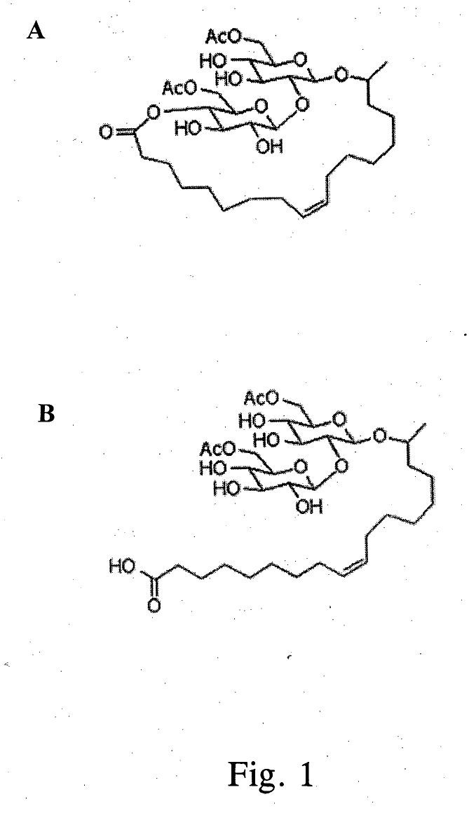

### DETAILED DESCRIPTION OF THE INVENTION

The present invention relates to methods for the production of open-chained sophorolipids, involving cultivating a yeast capable of producing open-chained sophorolipids in a culture medium containing fatty acid alkyl esters, and glycerol or carbohydrate (e.g., glucose) or mixtures thereof; wherein at least about 60% of the sophorolipids produced are open-chained sophorolipids. Preferably the culture medium does not contain free fatty acids or, if the culture medium already contains free fatty acids then no additional free fatty acids are added to the culture medium.

The present invention may utilize any yeast capable of producing open-chained sophorolipids, preferably Candida bombicola (e.g., ATCC 22214, NRRL Y-30816) or Candida apicola. Yeast strains may be tested for the production of open-chained sophorolipids by the methods described below.

In the present invention the fatty acid component of the culture medium may be the product of the direct transesterification of triacylglycerols or the product of the hydrolysis of triacylglycerols into free fatty acids and the esterification of said free fatty acids. Thus the culture medium may be biodiesel co-product stream (BCS), produced during the production of biodiesel, which is composed primarily of glycerol, acylglycerols, free fatty acids (FFA), and fatty acid alkyl (C₁₋₆) esters (principally fatty acid methyl esters (FAME)). Fatty acid methyl, ethyl and/or propyl esters may also be used as substrates for producing open chain SLs; methyl esters result in a large concentration of methylated fatty acid side chains on the SLs, whereas ethyl esters and propyl esters result in high concentrations of free acid, open chain SLs.

Production of biodiesel, and thus biodiesel co-product stream, is described in many publications, for example: Canakci, M., and J. Van Gerpen, Biodiesel Production from Oils and Fats with High Free Fatty Acids, Abstracts of the 92nd American Oil Chemists' Society Annual Meeting & Expo, p. S74 (2001); Freedman, B., et al., J. Am. Oil Chem. Soc., 61(10): 1638-1643 (1984); Graboski, M. S., and R. L. McCormick, Prog. Energy Combust. Sci., 24:125-164 (1998); Graboski, M. S., et al., The Effect of Biodiesel Composition on Engine Emissions from a DDC Series 60 Diesel Engine, Final Report to USDOE/National Renewable Energy Laboratory, Contract No. ACG-8-17106-02 (2000); Haas, M. J., et al., J. Am. Oil Chem. Soc., 77:373-379 (2000); Haas, M. J., et al., Energy & Fuels, 15(5):1207-1212 (2001); Haas, M. J., et al., Enzymatic Approaches to the Production of Biodiesel Fuels, in Kuo, T. M. and Gardner, H. W. (Eds.), Lipid Biotechnology, Marcel Dekker, Inc., New York, (2002), pp. 587-598); Holmberg, W. C., and J. E. Peeples, Biodiesel: A Technology, Performance, and Regulatory Overview, National SoyDiesel Development Board, Jefferson City, Mo. (1994); Krawczyk, T., INFORM, 7: 800-815 (1996); Mittelbach, M., and P. Tritthart, J. Am Oil Chem. Soc., 65(7):1185-1187 (1988); Peterson, C. L., et al., Applied Engineering in Agriculture, 13: 71-79 (1997); Sheehan, J., et al., Life Cycle Inventory of Biodiesel and Petroleum Diesel for Use in an Urban Bus, National Renewable Energy Laboratory, Report NREL/SR-580-24089, Golden, CO (1998); U.S. Pat. No. 2,383,601; U.S. Pat. No. 2,494,366; U.S. Pat. No. 4,695,411; U.S. Pat. No. 4,698,186; U.S. Pat. No. 4,164,506.

The most common method for making biodiesel practiced in U.S. industry at present involves the use of refined or partially refined soybean oil as feedstock. Other vegetable oils may also be used, but due to its low price and large-volume availability soy oil is essentially the only vegetable oil employed in this country. Animal fats and waste restaurant oils (as yellow and brown grease) may also be used in some U.S. facilities at some times, but soy oil is by far the predominant feedstock. The most frequently employed alcohol in biodiesel production in the U.S. is methanol (methyl alcohol). Other alcohols may be employed, but economics favors methanol. The most frequently employed catalysts in the U.S. are either sodium hydroxide dissolved in alcohol, or sodium alkoxide. The latter is prepared either by dissolving sodium metal in alcohol or by the electolysis of a sodium salt and subsequent reaction with alcohol. Potassium hydroxide can also be used, but due to its higher cost its use is sparse to nonexistent in the U.S. The present invention could utilize the products (e.g., glycerol, acylglycerols, free fatty acids, and fatty acid alkyl (C₁₋₆) esters) of such known methods.

U.S. Pat. No. 6,399,800 (incorporated by reference in its entirety) described a method for producing fatty acid alkyl esters from a feedstock (e.g. soy (soy soapstock), coconut, corn, cotton, flax, palm, rapeseed/canola, safflower, sunflower, animal fats, waste greases, or mixtures thereof), involving (a) saponifying the feedstock with an alkali (e.g., NaOH, KOH) to form a saponified feedstock, (b) removing the water from the saponified feedstock to form a dried saponified feedstock wherein the dried saponified feedstock contains no more than about 10% water, (c) esterifying the dried saponified feedstock with an alcohol (e.g., straight chain C₁₋₆ alcohol) and an inorganic acid catalyst (e.g., sulfuric acid) to form fatty acid alkyl esters, and (d) recovering said fatty acid alkyl esters. The present invention could utilize such a method after step (c) when the fatty acid alkyl esters have been produced and glycerol and fatty acids are still present.

U.S. Patent Application Publication No. 2003/0158074 (now U.S. Pat. No. 6,855,838, both incorporated by reference in their entirety) described a method for producing a lipid rich composition containing at least about 80% free fatty acids, involving reacting a feedstock with steam and alkali (e.g., NaOH, KOH) at a pH of about 10 to about 14 and further reacting the feedstock with steam and sulfuric acid at a pH of about 1 to about 2; the method optionally further involved esterifying the lipid rich composition with an alcohol (e.g., C₁₋₄ alcohol) and an inorganic acid catalyst (e.g., sulfuric acid, phosphoric acid, hydrochloric acid) to form a product containing fatty acid alkyl esters. The feedstock may be soy oil, coconut oil, corn oil, cotton oil, flax oil, palm oil, rapeseed/canola oil, safflower oil, sunflower oil, animal fats, waste greases, soy soapstock, coconut soapstock, corn soapstock, cotton soapstock, flax soapstock, palm soapstock, rapeseed/canola soapstock, safflower soapstock, sunflower soapstock, fully or partially hydrolyzed preparations made from soy, fully or partially hydrolyzed preparations made from coconut, fully or partially hydrolyzed preparations made from corn, fully or partially hydrolyzed preparations made from cotton, fully or partially hydrolyzed preparations made from flax, fully or partially hydrolyzed preparations made from palm, fully or partially hydrolyzed preparations made from rapeseed/canola, fully or partially hydrolyzed preparations made from safflower, fully or partially hydrolyzed preparations made from sunflower, fully or partially hydrolyzed preparations made from animal fats, fully or partially hydrolyzed preparations made from waste greases, or mixtures thereof. The present invention could utilize such a method since the product contains fatty acid alkyl esters, glycerol and fatty acids.

U.S. Patent Application Publication No. 2003/0229237 (incorporated by reference in its entirety) described a method for producing fatty acid alkyl esters, involving transesterifying a feedstock containing lipid-linked fatty acids with an alcohol (e.g., C₁₋₄ alcohol) and an alkaline catalyst (e.g., NaOH, KOH) to form fatty acid alkyl esters. The feedstock may be soy, coconut, corn, cotton, flax, palm, rapeseed/canola, safflower, sunflower, animal fats and oil, or mixtures thereof, where the feedstock has not been treated to release the lipid components of the feedstock. According to U.S. Patent Application Publication No. 2005/0020842 (incorporated by reference in its entirety), the feedstock may have been previously treated to release lipid components and the feedstock contains <about 20% lipids; for example the feedstock could be meat and bone meal or soy meal. The present invention could utilize such methods since the product contains fatty acid alkyl esters, glycerol and fatty acids.

Generally, the culture media may contain the following (in percent of the total culture media volume): about 8-about 17% (e.g., 8-17%) of glycerol and/or carbohydrate (e.g., glucose), preferably about 10-about 15% (e.g., 10-15%), more preferably about 12-about 13% (e.g., 12-13%); about 3-about 10% (e.g., 3-10%) alkyl esters (e.g., methyl esters), preferably about 5-about 8% (e.g., 5-8%), more preferably about 6-about 7% (e.g., 6-7%). Generally, the above components are all delivered to the fermentation reaction in three installments (e.g., at the beginning, at about 24 h, and at about 48 h).

The culture medium may incorporate a mineral nitrogen source (in the form of ammonium ions) and/or organic nitrogen, e.g. in the form of amino acids such as in particular yeast extract, soybean peptone, casein hydrolysates, maize/corn maceration liqueur, wheat gluten hydrolysates, and meat extracts. The addition of mineral elements such as e.g. potassium, sodium, magnesium and oligoelements such as iron, manganese, molybdenum in the form of their salts (sulphates, phosphates, chlorides) may also make it possible to further improve growth of the yeast. For optimum production yields, the medium must also include at least one sugar or carbohydrate such as glucose or a compound that can be readily metabolized to glucose (e.g., glycerol) by the normal enzymatic pathways available to the yeast.

The culture conditions are generally as follows: temperature of about 18° to about 35° C. (e.g., 18°-35° C.), preferably about 20′ to about 30° C. (e.g., 20°-30° C.), more preferably about 22′ to about 28° C. (e.g., 22°-28° C.); pH of about 3 to about 8 (e.g., pH 3-8), preferably pH of about 3 to about 5 (e.g., pH 3-5), more preferably pH of about 3.5 to about 4 (e.g., pH 3.5-4); pressure of about 1 to about 5 bar (e.g., 1-5 bar) and preferably about 1 to about 2 bar (e.g., 1-2 bar); incubation time of about 3 days to about 9 days (e.g., 3-9 days), preferably about 4 days to about 8 days (e.g., 4-8 days), more preferably about 6 days to about 7 days (e.g., 6-7 days).

Throughout the production time, the pH may be checked and regulated to a desired value within the range described hereinbefore by e.g. adding potash or soda solution.

Generally, at least about 60% (e.g., at least 60%) of the sophorolipids produced in the present invention are open-chained sophorolipids; preferably at least about 65% (e.g., at least 65%), more preferably at least about 70% (e.g., at least 70%), more preferably at least about 75% (e.g., at least 75%), more preferably at least about 80% (e.g., at least 80%), most preferably at least about 85% (e.g., at least 85%). Generally, when grown on biodiesel coproduct stream, at least about 60% (e.g., at least 60%) of the sophorolipids produced in the present invention are open-chained sophorolipids; preferably at least about 65% (e.g., at least 65%), more preferably at least about 70% (e.g., at least 70%), and most preferably at least about 75% (e.g., at least 75%). When synthesized from mixed substrate fermentations made up of glycerol and soy fatty acid alkyl esters, including methyl soyate, ethyl soyate or propyl soyate, preferably at least about 60% (e.g., at least 60%) of the sophorolipids are open-chained sophorolipids, more preferably at least about 65% (e.g., at least 65%), more preferably at least about 70% (e.g., at least 70%), more preferably at least about 75% (e.g., at least 75%), more preferably at least about 80% (e.g., at least 80%), and most preferably at least about 85% (e.g., at least 85%). Thus the sophorolipids produced in the present invention will primarily be open-chained sophorolipids, but there will also be some lactonized or lactone form of the SLs. Generally, the sophorolipids will have the following formulas: 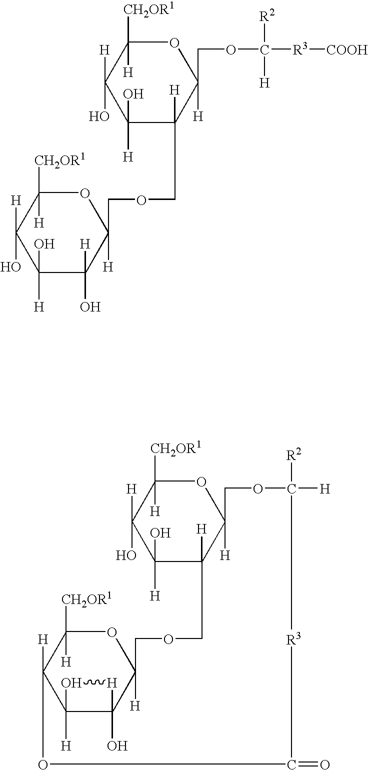
in which R¹ represents hydrogen or an acetyl group and R² hydrogen or an alkyl group having 1 to 9 carbon atoms (e.g., CH₃), when R³ is a saturated hydrocarbon radical with 7 to 16 carbon atoms (e.g., 13-15 carbon atoms), or R² represents hydrogen or a methyl group, when R³ is an unsaturated hydrocarbon radical with 13 to 17 carbon atoms (e.g., 13-15 carbon atoms). Preferably, when the culture media contain fatty acid methyl esters, an alkyl group (e.g., methyl) is attached at the carboxyl end of the fatty acid component of the SLs because we have found that the presence of the alkyl group (e.g., methyl) inhibits the lactonization of the molecules and ultimately increases the amount of open chain sophorolipids in the present process.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

The following examples are intended only to further illustrate the invention and are not intended to limit the scope of the invention as defined by the claims.

### EXAMPLES

### Example 1

Strain information and inoculum preparation: Candida bombicola ATCC 22214 was obtained from American Type Culture Collection (Manassas, Va.). The inocula for all of the SL production cultures were prepared in Candida Growth Media (CGM) which contained glucose (10% w/v), yeast extract (1% w/v), and urea (0.1% w/v) (Asmer, H-J., et al., J. Am, Oil Chem. Soc., 65: 1460-1466 (1988)), and were grown at 26° C. with shaking at 250 rpm. Frozen stock cultures were prepared by inoculating 100-mL of CGM (contained in a 250-mL Erlenmeyer flask) with a loopful of C. bombicola and grown for 24 h. Ten mL of this culture was then inoculated into 500-mL of CGM in a 1-L Erlenmeyer flask and the culture allowed to grow for an additional 48 h. The culture was then divided into 200 mL volumes and the cells were aseptically collected by centrifugation (16,000×g; 10 min; 4° C.). Fifty mL of a sterile aqueous glycerol solution (10% v/v) was then used to resuspend each cell pellet and the cell suspensions were frozen in a dry ice/acetone bath and stored at −80° C. until needed.

Sophorolipid production: Production of sophorolipids was performed in 2 L volumes in a 2.5-L capacity bench-top fermentor (BIOFLO III Batch/Continuous Bioreactor, New Brunswick, N.J.). The SL production medium contained the following (in g/L): glycerol (99% pure, Sigma-Aldrich Chemical Co., St. Louis, Mo.) or BCS (Ocean Air Environmental, Lakeland Fla.), 100 (composition of BCS described below); yeast extract, 10; and urea, 1. The pH was brought to pH 6.0 with concentrated HCl and the media was sterilized by autoclaving. The glycerol or BCS was added as the carbon source for the fermentations in place of the glucose present in the CGM described above. A single 50 mL frozen stock culture was thawed and used to inoculate the 2 L media. The fermentations were carried out at 26° C., 700 rpm, 2 L air/min, and an initial pH of 6.0. After 24 h, an additional 100 mL of glycerol or BCS was added to the culture and the pH set to maintain at 3.5. At the 48 h time-point, an additional 100 mL of glycerol or BCS was added to the culture, and the fermentations were allowed to continue for an additional 5 days.

Sophorolipid recovery and purification: To isolate the SLs, the entire culture (cells and broth) was divided into 6 portions and lyophilized to dryness (˜2 days). The dried residues were placed into six 1-L Erlenmeyer flasks, and each portion extracted with 500 mL of ethyl acetate by shaking at 250 rpm, 30° C. for 5 days. The extraction mixture was filtered through Whatman No. 2 filter paper, and the solid residues were rinsed an additional 2 times with ethyl acetate (500 mL each time) to maximize SL recovery. The combined filtrate was concentrated by evaporation and added to 1 L of a mixture of hexane/petroleum ether (90/10 v/v) to precipitate out the pure SLs. After vacuum drying in a desiccator, the purified SLs were accurately weighed to obtain the product yield.

BCS composition: BCS was assayed for glycerol, fatty acid soaps (as FFA), and FAME. Glycerol content (in mg/dL) was determined using the Glycerol-SL assay (Diagnostic Chemicals Limited, Charlottetown, P.E.I., Canada) according to manufacturer's specifications. FFA and FAME content were determined gravimetrically as follows: The alkaline BCS (100 g) was acidified to pH 1 with concentrated HCl and extracted with three 50-mL volumes of hexane. The hexane layers were combined into a tared flask, evaporated under nitrogen, and dried in vacuo (25 in Hg) for 24 h to a constant weight. The flask was then reweighed and the amount of hexane-solubles (FFA and FAME) was calculated by difference from the starting weight. The total concentration of FFA and FAME was determined by dissolving a known amount of hexane-solubles back into hexane and using an established high performance liquid chromatography (HPLC) procedure (Foglia, T. A., and K. C. Jones, J. Liq. Chrom. Related Technol., 20: 1829-1838 (1997)).

FFA/FAME compositional analysis: While the HPLC procedure described above was used to determine the total amount and ratio of FFA and FAME in the BCS, the specific FFAs and FAMEs present in the hexane soluble fraction of BCS were determined by gas chromatography/mass spectrometry (GC/MS). Samples were dissolved in hexane at a concentration of 10 mg/mL and silylated by reacting 10 μL of each sample with 250 μL of N,O-bis(trimethylsilyl)-trifluoroacetimide (BSTFA; Pierce Biotechnology Inc., Rockford, Ill.) and 200 μL of pyridine. The mixtures were heated at 70° C. for 30 min and allowed to cool to room temperature. Finally, an additional 150 μL of hexane was added to each sample and the samples analyzed by GC/MS. Samples were injected (1 μL) into a Hewlett Packard (HP, Wilmington, Del.) gas chromatograph (GC) model 5890 Series II Plus equipped with a capillary inlet and an HP Mass Selective Detector (MSD) model 5972 Series set to scan from m/z 40 to m/z 550 at a rate of 1.5 scans/s. The capillary column (30 m×0.25 mm) was coated with 0.25 μm of 5% cross-linked phenyl methyl silicone (HP-5MS). The oven temperature was programmed from 80° C. (1 min) to 230° C. (10 min) at 10° C./min. The injector port temperature was 250° C. in the splitless mode and the detector transfer line at 280° C.

Sophorolipid Analysis: The SL content was determined as described previously (Nuñez, A., et al., Chromatographia, 53: 673-677 (2001)). Generally, the SL mixtures were separated by HPLC with a Waters 2690 Separation Module (Waters Co. Milford, Mass.) using a 15 cm×2.1 mm Symmetry C18 3.5 μm column. The linear gradient elution used was as follows: water:acetonitrile (0.5% formic acid): acetonitrile (50:10:40) hold for 5 min to a final composition of acetonitrile (0.5% formic acid): acetonitrile (10:90) over 25 min with a total running time of 40 min. The flow rate was 0.25 mL/min. The effluent was connected to a Micromass ZMD mass spectrometer with an APCI probe (Waters Co.) set to the positive mode to scan from m/z 200 to m/z 900 at 2 s per scan. Corona pin voltage was tuned to 3.8 kV, sample cone 20 V and extraction cone 2 V for detection of fragments and molecular ions.

Results and Discussion: We tested BCS as a substrate for the synthesis of open-chained SLs in an attempt to find a new outlet for BCS. In addition, we postulated that the presence of a methyl group on the carboxyl end of the fatty acid would hinder lactonization of the SL and result in an increased concentration of open-chain structures. This would permit further post-synthetic modification of the fatty acid side-chain without the need to open the lactone ring. Compositional analysis revealed that the BCS used in this study was composed of 40% glycerol, 34% hexane-solubles (made-up of 92% FFA/FAME and 6% mono- and diacylglycerols), and 26% water. GC/MS analysis of the hexane-soluble fraction indicated the presence of substantially higher amounts of FFA compared to FAME. Specifically, the hexane-soluble fraction was composed of the following ratios of FFA and FAME (in mol %): myristic acid, 1; palmitic acid, 18; stearic acid, 13; oleic acid, 25; linoleic acid, 25; and the methyl esters of palmitic acid, 2; stearic acid, 1; oleic acid, 7; and linoleic acid, 7. The higher concentrations of FFA compared to FAME (on a mol % basis) in the BCS indicated that while the recovery rate of the biodiesel (i.e., fatty acid alkyl esters) from the BCS may have been less than optimal, the incomplete conversion of the starting oil to methyl esters probably caused the large hexane soluble-fraction in the BCS. In addition, the ratio of oleic acid to linoleic acid in the BCS reflected the soybean-oil origin of the biodiesel.

C. bombicola grew and produced SLs from both pure glycerol and BCS. Specifically, pure glycerol resulted in low yields of SLs (˜9 g/L) while the use of BCS increased the SL yields to 60 g/L. The fermentations used in this study consisted of an initial 10% (w/v) carbon source concentration prior to inoculation. This translated into approximately 4% (w/v) glycerol in the BCS fermentations compared to 10% (w/v) glycerol in the pure glycerol fermentations. This large discrepancy was enough to affect the productivity of the culture. In fact, the cell yields from the pure glycerol fermentations were 8.7±2.1 g/L compared to 42.5±3.0 g/L from the BCS fermentations, indicating that higher glycerol concentrations had a deleterious effect on the growth of C. bombicola.

We previously reported an LC/APCI-MS method that allowed for the separation and characterization of SLs (Nuñez, A., et al., Chromatographia, 53: 673-677 (2001)). The mass spectrum generated by APCI for either the lactone or free acid form of SL had a protonated molecular ion (M+H)⁺ followed by a series of ions with decreasing intensity corresponding to the multiple loss of H₂O molecules. The SL spectrum typically showed a characteristic mass loss of 204 amu from the molecular ion, which corresponded to a C6 acetylated hexose ring. The lack of acetyl groups on the sophorose ring was reflected by this ion. Both SL forms gave a fragment ion for the protonated hydroxyl fatty acid, which was at m/z 299 for the C18:1 SL, providing evidence of the fatty acid moiety. This ion was more intense in the free acid form than the lactone. Also, the non-lactonized free acid form presented a characteristic ion at m/z 409 which corresponded to the disaccharide fragment. Since the m/z 409 ion was independent of the fatty acid moiety and had a negligible intensity in the lactonized form, the extracted ion chromatogram resulting from this ion allowed the identification of the non-lactonized form eluting from the column.

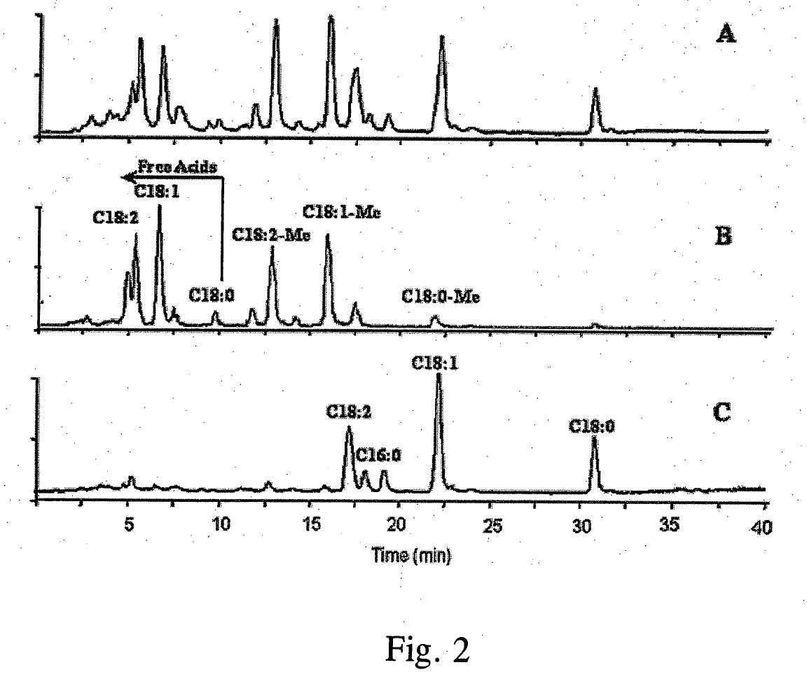

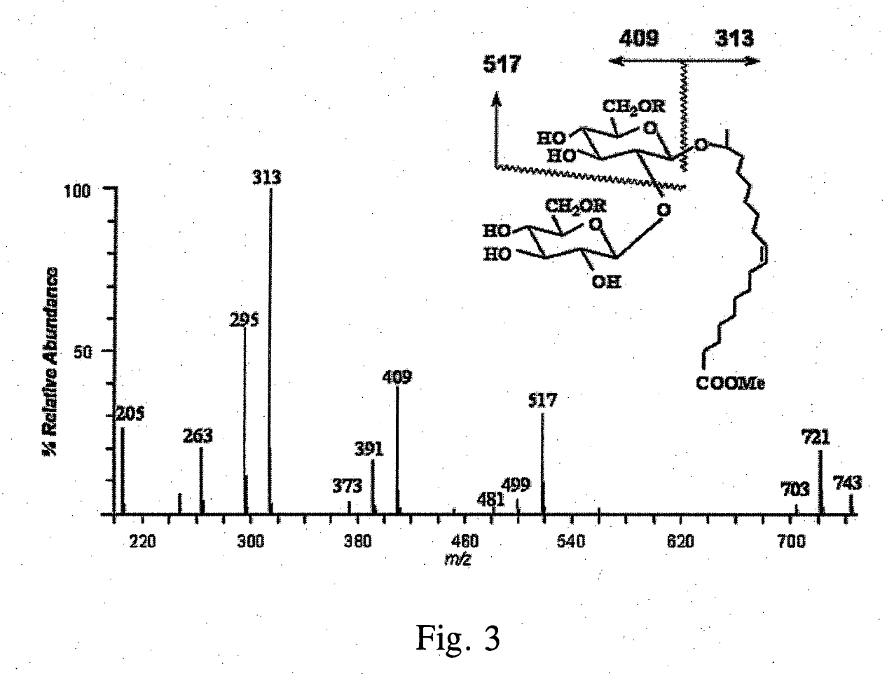

The fatty acid moiety in the SL structure can be influenced by the fatty acid content of the oil used as the carbon source, but in general the lactone form with a C18:1 fatty acid moiety was secreted as the major product (Nuñez, A., et al., Chromatographia, 53:673-677 (2001)). When BCS was used as the carbon source, the LC/APCI-MS chromatogram had the elution profile shown in FIG. 2A, which was more complex than those previously observed. Extraction of the ion at m/z 409 from the total ion current chromatogram indicated that non-lactonized forms of the fatty acid were responsible for the different chromatographic profile. The peaks eluting in the first 10 minutes had spectra consistent with free fatty acids as labeled in FIG. 2B. The second, more intense peak eluting at about 16 minutes (FIG. 2B) had the APCI spectrum shown in FIG. 3. The spectrum had a base peak at m/z 313 consistent with a methylated C18 hydroxy fatty acid ion fragment, followed by double bond formation at the hydroxy group and the loss of the methoxy group to generate the ions at m/z 295 and 263, respectively. The ions at m/z 721 and 743 confirmed the [M+H]⁺ and [M+Na]⁺, respectively, corresponding to a SL structure bearing a methylated C18:1 fatty acid that generates the ion at m/z 703 after the loss of water. The loss of mass 204 from the molecular ion to generate the ion at m/z 517 was consistent with the loss of an acetylated hexose ring. The chromatographic peaks eluting between 10 and 22 minutes had spectra that were consistent with non-lactonized methyl ester forms and were labeled accordingly in FIG. 2B. Plotting of the mass chromatogram in the range of 600-700 amu produced the chromatogram shown in FIG. 2C, whose spectra of the individual peaks were consistent with lactone forms and were labeled according to the fatty acid moiety.

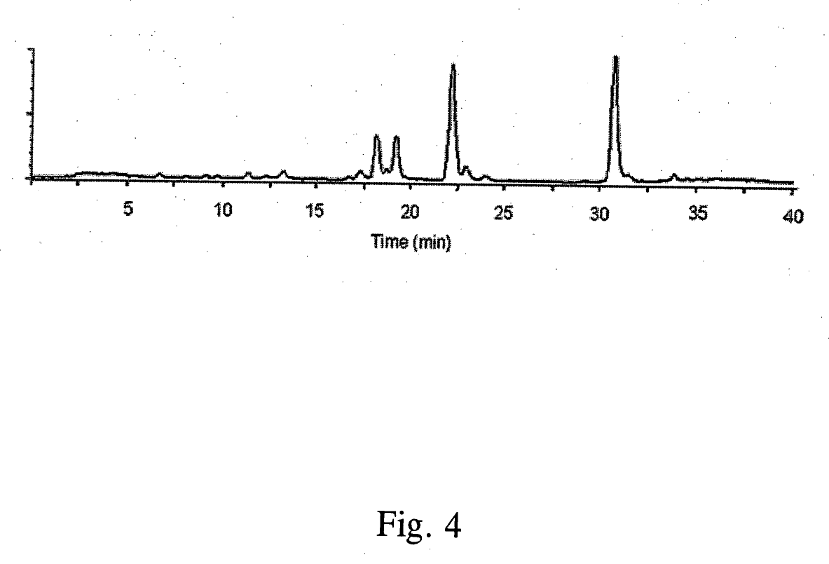

When the yeast was grown with glycerol as the carbon source, the product analysis revealed the formation of the lactone forms as the main products with negligible amounts of non-lactonized SL as shown in FIG. 4. In an effort to maximize the open-chain conformation of SLs derived from glycerol based fermentations, FAME in the form of methyl soyate were added to the media at a concentration of 12.5 g/L, thus mimicking the same ratio of glycerol to hexane-soluble fraction present in the BCS. These fermentations resulted in SL yields of 43 g/L. Upon LC/APCI-MS analysis, it was evident that the inclusion of FAME produced a chromatogram with peaks having elution times and spectra consistent with the chromatogram obtained for the BCS (FIG. 2). The total composition of the SL derived from pure glycerol, BCS, and glycerol+FAME are given in Table 1. Results showed that there was no benefit to spiking the fermentation media with FAME in comparison to the use of BCS alone. In both cases the open-chain conformation was the most prevalent, accounting for 75% of the SLs synthesized from BCS vs. 69% of the SLs synthesized from glycerol+FAME with oleic acid and linoleic acid predominating. In comparison, the SLs derived from pure glycerol were 99% lactone with the majority of the fatty acids either palmitic, stearic or oleic.

Thus the content of BCS makes it a viable substrate for the microbial synthesis of open-chained SLs. As mentioned earlier, SLs have a number of potential applications and can be synthesized in large quantities. In addition, while pure glycerol resulted in SLs that were almost entirely in the lactone conformation, the presence of FFAs and FAMEs in BCS induced the formation of SLs in the open form. The high concentrations of open form SLs will benefit subsequent post-synthetic modification of the fatty acid side-chain without the need for ring opening reactions, thus lowering cost and benefiting the biodiesel market. The ability to use the BCS as a feedstock for open-chained SL synthesis will provide an outlet for this residual material, thus helping to stimulate growth in the biodiesel market and the utilization of agricultural fats and oils from which the biodiesel was synthesized.

### Example 2

### Transesterification of Soy Oil

Methyl-, ethyl- and propyl-soyates were prepared by alkali-catalyzed transesterification (Freedman, B., et al., J. Am. Oil Chem. Soc., 61: 1638-1643 (1984)) using a 6:1 molar ratio of alcohol to soy oil (Sigma Chemical Co.). The fatty ester profile of the soy oil used in this study was as follows: palmitic acid, 11%, stearic acid, 3%, oleic acid, 21%, linoleic acid, 57%, and linolenic acid, 8%. Specifically, assuming an average molecular weight of 872 amu for soy triacylglycerol, 436 g of soy oil was added to each of 3 separate 1-L finned Erlenmeyer flasks and 96 g of methanol, 138 g of ethanol and 180 g of propanol were added to the flasks, respectively. Dry NaOH pellets (4.36 g/flask) were added to each flask to induce alkaline conditions and each flask was capped to inhibit alcohol evaporation and stirred briskly overnight (the propanol-containing flask was heated at 85° C. for the duration of the reaction to help dissolve the NaOH pellets). At 24 h, the reaction temperatures were brought back to ambient and the pH was neutralized with HCl. The excess alcohol was then removed by rotary evaporation. Five-hundred mL of 0.5M NaCl was added to each flask to inhibit emulsion development and each flask was gently shaken at 100 rpm, 30° C. for 2 h to separate the glycerol phase from the ester phase. The top layer of each flask (ester phase) was decanted from the aqueous layer and placed into separate jars containing anhydrous Na₂SO₄ to remove residual water. The ester fractions were stored at room temperature until needed as fermentation substrates.

Strain information and SL production: The yeast Candida bombicola ATCC 22214 was used as the producer strain in all experiments. The inocula were prepared in a glucose (10% w/v)/yeast extract (1% w/v)/urea (0.1% w/v) medium (GYU medium) as described by Asmer et al. (Asmer H.-J., et al., J. Am. Oil Chem. Soc., 65: 1460-1466 (1988)) and were grown at 26° C. with shaking at 250 rpm overnight. Cells were frozen in 10% glycerol and stored in 50 mL volumes at −80° C. until needed. Sophorolipid synthesis was carried out in 2-L volumes in a 2.5-L capacity Bioflo III batch/continuous bench-top bioreactor (New Brunswick Scientific). The basal production medium contained the following (in g/L): glycerol (commercial glycerol was used but the glycerol fraction of the transesterification reaction could also be used), 100; yeast extract, 10; and urea, 1. The pH was adjusted to pH 6.0 with concentrated HCl and the media were sterilized by autoclaving. After cooling, 45 mL of filter (0.45μ pore-size) sterile methyl-soyate (fatty acid esters prepared by transesterification of soy oil with methanol), ethyl-soyate or propyl-soyate was added and the fermenter inoculated using a single 50 mL frozen stock culture. All fermentations were carried out at 26° C., 700 rpm and 2-L air/min. After 48 h, 100 mL of glycerol and 45 mL of alkyl esters were added to the fermentations. At the 72 h time-point, an additional 50 mL of glycerol and 45 mL of alkyl esters were added to the cultures, and the fermentations allowed to continue for an additional 4 days (total fermentation time=7 days).

SL recovery and purification: To isolate the SLs, the entire culture (cells and broth) was divided into 6 portions and lyophilized to dryness. The dried residues were placed into 6 1-L Erlenmeyer flasks, and each portion extracted with 500 mL of ethyl acetate by shaking at 250 rpm, 30° C. for 3 days. The mixture was filtered through Whatman No. 2 filter paper, and the solid residues rinsed with ethyl acetate (2×500 mL) to maximize recovery. The combined filtrates were concentrated by rotary evaporation and added to 1-L of hexane to precipitate the pure SLs. After vacuum drying in a desiccator, the purified SLs were accurately weighed to obtain the product yield. In all cases purity was determined to be greater than 95%.

Ester formation and SL compositional analysis: Alkyl ester formation efficiency was determined by HPLC on a Hewlett Packard HP1050 system with a 10 cm×3 mm LiChrosorb Diol column (Varian Inc.) with a pore size of 100 Å. The isocratic mobile phase was 1000:1 hexane:acetic acid with a flow rate of 0.5 mL/min and a total run time of 15 min. Under these conditions the fatty acid esters did not speciate but were effectively separated from any sterol esters, free fatty acids or triacylglycerols remaining in the sample. As noted above, the fatty acid esters did not speciate, in other words the HPLC method did not allow for the separation of the individual fatty acids in the soy oil but rather all of the alkyl esters regardless of the specific fatty acid eluted together; we were able to see the production of fatty acid alkyl esters but were unable to divide them up into oleic acid alkyl esters, linoleic acid akyl esters, etc.

Sophorolipid content was determined by HPLC using a Waters 2690 Separation Module (Waters Co.) equipped with a 15 cm×2.1 mm Symmetry C18 3.5 μm column. The linear gradient elution was as follows: water:acetonitrile (0.5% formic acid): acetonitrile (50:10:40) hold for 5 min to a final composition of acetonitrile (0.5% formic acid): acetonitrile (10:90) over 25 min with a total running time of 40 min. The flow rate was 0.25 mL/min. The effluent was connected to a Micromass ZMD mass spectrometer with an APCI probe (Waters Co.) set to the positive mode to scan from m/z 200 to m/z 900 at 2 seconds per scan. Corona pin voltage was tuned to 3.8 kV, sample cone 20 V and extraction cone 2 V for detection of fragments and molecular ions.

Results and Discussion: We showed above that SLs can be synthesized in relatively large yields (˜60 g/L) by fermentation of C. bombicola on the biodiesel coproduct stream (containing glycerol and small concentrations of mono- and diacylglycerols, free fatty acids and fatty acid methyl esters) and that those SLs were primarily in the open-chain form. We postulated that the tendency for the open-chain structure was the result of the methyl ester, which repressed lactone formation.

C. bombicola was grown in the presence of glycerol and either methyl-, ethyl- or propyl-soyate to determine the feasibility of SL production from these substrates and their potential influence on the chemical structures of any SLs produced. Glycerol was used as a co-substrate because it supports yeast growth and is expected to be produced in large amounts over the next few years as a coproduct of an expanding biodiesel industry. C. bombicola grew and produced SLs in yields of 46±4 g/L, 42±7 g/L and 18±6 g/L from methyl soyate (Me-Soy), ethyl soyate (Et-Soy), and propyl soyate (Pro-Soy), respectively. We showed above that pure glycerol supported growth and low level production (˜9 g/L) of SLs by C. bombicola. Addition of the soy esters into the fermentation media increased the SL productivity by a minimum of 100% (w/propyl soyate) and a maximum of 511% (w/Me-Soy).

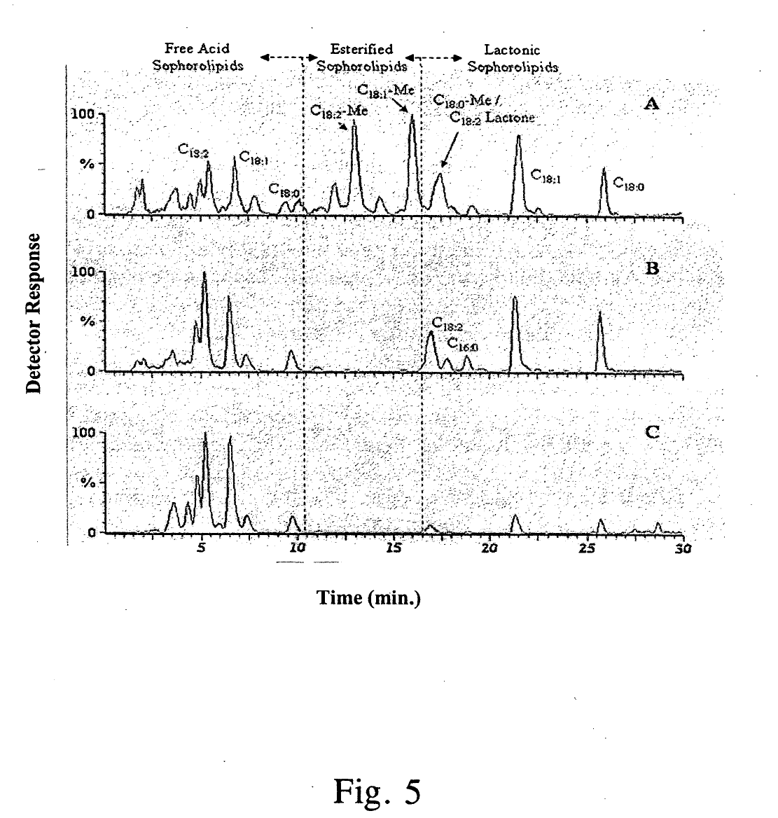

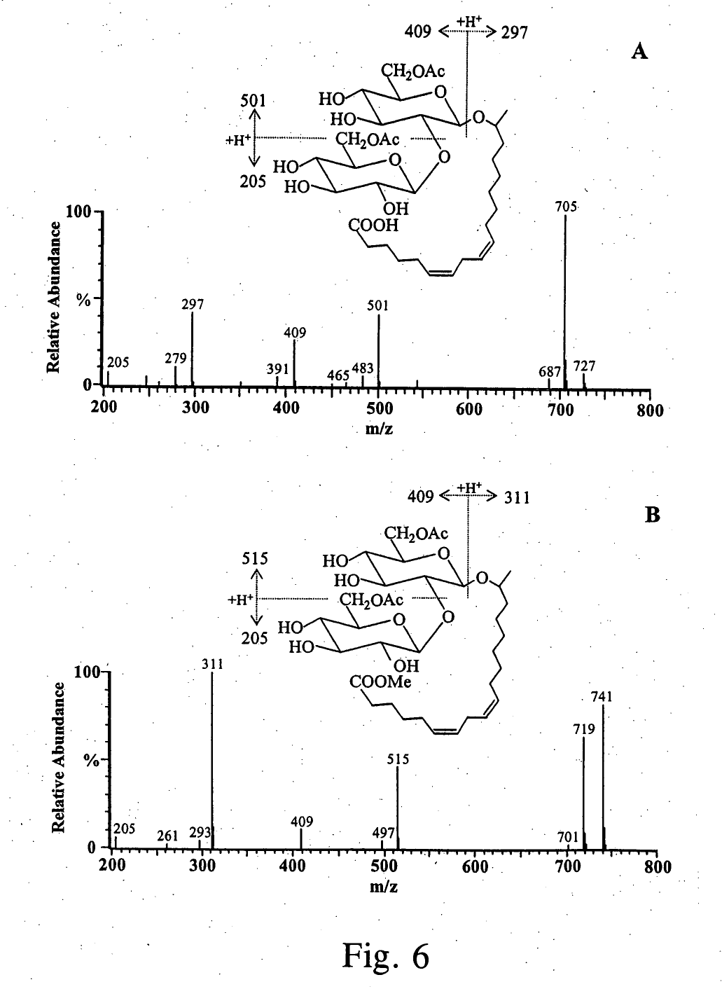

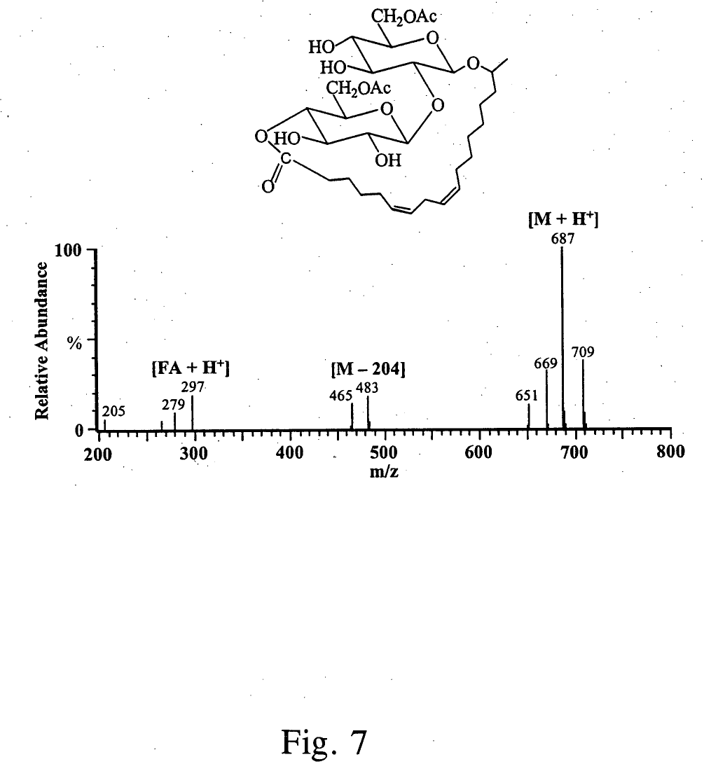

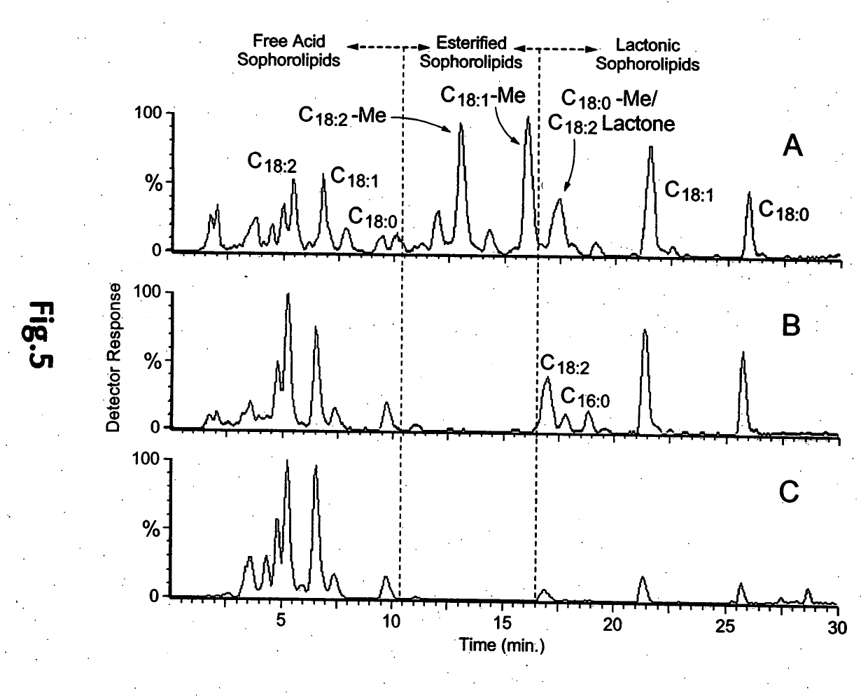

The ratios of the SL structural variants created from each ester were determined using LC/APCI-MS. The total ion chromatograms (TIC) of the SLs produced from Me-Soy, Et-Soy, and Pro-Soy are shown in FIG. 5. The total number of peaks in each TIC revealed that each ester resulted in a large number of structural variants. By counting the total number of peaks in each TIC, it was clear that the SLs produced from glycerol and Me-Soy (FIG. 5A) contained the largest number of structural variants when compared to the other esters. The fatty ester profile of the soy oil used in this study was as follows: palmitic acid, 11%, stearic acid, 3%, oleic acid, 21%, linoleic acid, 57%, and linolenic acid, 8%. Because of the high oleic acid and linoleic acid content of the esters, along with the preference for oleic acid in SL production, it was expected that a large number of the SL molecules would be linked to either oleic or linoleic acid residues. The APCI-mass spectra corresponding to SLs containing linoleic acid in the free-acid open-chain form and the methyl esterified open-chain form can be seen in FIG. 6 and that of the lactone form in FIG. 7. Specifically, FIG. 6A shows the APCI spectrum of the diacetylated C18:2 free-acid SL, which eluted from the column at roughly 5 min (see FIG. 5). The spectrum had a base peak at m/z 705 which corresponded to the protonated molecular ion (M+H⁺). The peak at m/z 727 indicated the addition of sodium to the molecular ion (M+Na⁺). The loss of water from the M+H⁺ ion generated the peak at m/z 687, while loss of an acetylated hexose sugar (204 amu) resulted in the peak at m/z 501 with a sequential loss of 2 water molecules resulting in the peaks at m/z 483 and m/z 465. This spectrum also contained a peak at m/z 409, which corresponded to the fatty acid-free disaccharide fragment. This peak was characteristic of all open-chain SL (whether free-acid or esterified forms) and was independent of the fatty acyl group associated with the SL. The protonated hydroxy fatty acid peak at m/z 297 was indicative of linoleic acid (C_{18:2}) and the peak at m/z 205 corresponded to a protonated acetylglucose moiety.

FIG. 6B shows the APCI-mass spectrum of the C_{18:2} methyl ester SL, which eluted from the column at about 13 min (see FIG. 5). In this spectrum the M+H⁺ ion was at m/z 719 accompanied by an M+Na⁺ ion at m/z 741. The molecular ion in FIG. 6B was 14 amu larger than that in FIG. 6A, which indicated the presence of a methyl group esterified at the carboxyl terminus of the fatty acid. As in the free-acid form, the peak at m/z 409 (which corresponded to the diacetylated disaccharide) indicated that the molecule was in the open-chain form. The ions at m/z 311 (which corresponded to the protonated linoleic acid methyl ester) and m/z 515 (which corresponded to the acetylated monosaccharide form of the SL) also confirmed the existence of a methylated, open-chain SL.

FIG. 7 shows the APCI-spectrum of the diacetylated C_{18:2} lactone that eluted from the column at approximately 17 min. This spectrum showed a base peak at m/z 687, which corresponded to the M+H⁺. Peaks at m/z 669 and m/z 651 corresponded to the sequential loss of water, and a peak at m/z 709 corresponded to the M+Na⁺. The loss of an acetylated hexose ring resulted in the peak at m/z 483 with the loss of water (m/z 465). Evidence for the hydroxy fatty acid structure was obtained from the peak at m/z 297, which after the loss of water gave a peak at m/z 279.

Based on elution times and fragmentation patterns, the chemical structures that correlated to each chromatographic peak were determined. Moreover, because the SL lactone forms lacked ions at m/z 409, it was possible to determine which chromatographic peaks corresponded to open-chain SLs simply by extracting the m/z 409 ions from the chromatograms. In addition, plotting of the mass chromatogram in the range of 600-700 amu produced chromatograms whose individual spectra were consistent with lactone forms (data not shown). By filtering the TIC in this manner it was determined that only 1 peak in the TIC was the result of co-eluting molecules. These overlapping peaks were present only in the TIC of SL derived from Me-Soy (FIG. 5A) eluting at roughly 17.5 min and represented SL containing linoleic acid in the lactone form and SL in the open-chain form containing stearic acid methyl ester. Table 2 lists the open-chain content and distribution of the SLs produced from the methyl-, ethyl- and propyl-soyates. Because of the high content of linoleic acid in soy oil, it was not surprising to find substantial amounts of linoleic acid present in the SLs. In addition, oleic acid, which is a preferred fatty acid component in SLs, was also a major component of the SLs regardless of the ester used as substrate. Interestingly, only the SLs derived from Me-Soy resulted in esterified fatty acid side chains (approximately 42 mol %), which eluted from the column between 11 and 16.5 min. The SLs derived from Et-Soy and Pro-Soy showed no esterified side chains but higher concentrations of free-acid open-chain forms. Specifically, those SLs produced from Et-Soy were 43 mol % in the free-acid open-chain form while SLs synthesized from Pro-Soy were 80 mol % in the free-acid open-chain form with the majority linked at the ω-1 position of the fatty acid chain. These results suggested that C. bombicola possessed a means (which was most active against ethyl and propyl groups but only partially active against methyl groups) by which the ester groups can be removed from the molecules. This was shown by the fact that neither the ethyl ester-grown nor the propyl ester-grown SLs contained esterified products. The fact that C. bombicola had lipase activity (as evidenced by its ability to use intact triacylglycerols) may explain this ester hydrolysis activity. The only SLs that showed any ester side-chains (59% of the total open-chain form) were the methyl ester-grown SLs. The only SLs that showed greater than 50 mol % lactone were those produced from Et-Soy (Table 3).

In conclusion, it was demonstrated that the use of Pro-Soy as a carbon substrate for the production of SLs by C. bombicola resulted in the lowest SL yields but also resulted in the highest concentration (80%) of free-acid open-chain SLs. While Me-Soy improved the amount of open-chain SLs, 59% of those open-chain molecules remained esterified, thus restricting the possibilities for chemical modification at the carboxyl terminus of the fatty acid. Et-Soy resulted in more free-acid open-chain SL (43 mol %) but less than half of these molecules were in the open-chain form. Thus, in view of the above, fatty acid methyl esters (FAME), fatty acid ethyl esters (FAEE) and fatty acid propyl esters (FAPE) can all be used in conjunction with glycerol as substrates to increase the amount of open-chain SLs. In Example 1 we described the use of the biodiesel coproduct stream as a substrate to boost open chain SL production, Example 2 describes the use of the alkyl esters themselves as substrates with glycerol to further boost open-chain, free acid SLs.

All of the references cited herein are incorporated by reference in their entirety. Also incorporated by reference in their entirety are the following references: Ashby, R. D., et al., J. Polym. Environ., 12:105-112 (2004); Ashby, R. D., et al., J. Am. Oil Chem. Soc., 82: 625-630 (2005)); Braunegg, G., et al., Macromol. Symp., 144:375-383 (1999); Freedman, B., et al., J. Am. Oil Chem. Soc. (JAOCS), 61: 1638-1643 (1984); Guilimanov, V., et al., Biotechnol. Bioengin., 77: 489-494 (2002); Otto, R. T., et al., Appl. Microbiol. Biotechnol., 52: 495-501 (1999); Rau, U., et al., Biotechnol. Lett., 18: 149-154 (1996); Rau, U., et al. Biotechnol. Lett., 21: 973-977 (1999); Solaiman, D. K. Y., et al., Inform, 15: 270-272 (2004); Taidi, B., et al., Appl. Microbiol. Biotechnol., 40:786-790 (1994).

Also incorporated by reference in their entirety are the following U.S. Pat. Nos. 5,616,479; 5,900,366; 5,879,913.

Thus, in view of the above, the present invention concerns (in part) the following:

A method for the production of open-chained sophorolipids, said method comprising (or consisting essentially of or consisting of) cultivating a yeast capable of producing open-chained sophorolipids in a culture medium comprising (or consisting essentially of or consisting of) fatty acid alkyl esters, at least one member of the group consisting of glycerol, carbohydrate, and mixtures thereof; wherein at least about 60% of the sophorolipids produced are open-chained sophorolipids.

The above method, wherein said fatty acid alkyl esters are fatty acid alkyl (C₁₋₆) esters.

The above method, wherein said yeast is selected from the group consisting of Candida bombicola, Candida apicola, and mixtures thereof.

The above method, wherein said yeast is Candida bombicola.

The above method, wherein said yeast is Candida bombicola NRRL Y-30816

The above method, wherein at least about 65% of the sophorolipids produced are open-chained sophorolipids.

The above method, wherein at least about 70% of the sophorolipids produced are open-chained sophorolipids.

The above method, wherein at least about 75% of the sophorolipids produced are open-chained sophorolipids.

The above method, wherein at least about 80% of the sophorolipids produced are open-chained sophorolipids.

The above method, wherein at least about 85% of the sophorolipids produced are open-chained sophorolipids.

The above method, wherein said fatty acid alkyl esters are fatty acid methyl esters, ethyl esters, propyl esters, or mixtures thereof. The above method, wherein said fatty acid alkyl esters are fatty acid methyl esters or fatty acid ethyl esters or fatty acid propyl esters or any combination thereof.

The above method, wherein said culture medium contains glycerol.

The above method, wherein said culture medium contains carbohydrate.

The above method, wherein said culture medium contains glycerol but not carbohydrate.

The above method, wherein said culture medium comprises (or consists essentially of or consists of) fatty acid alkyl esters, carbohydrate, and optionally free fatty acids.

The above method, wherein said culture medium comprises (or consists essentially of or consists of) fatty acid alkyl esters, glycerol, and optionally free fatty acids.

The above method, wherein said culture medium does not contain free fatty acids.

The above method, wherein said culture medium comprises (or consists essentially of or consists of) fatty acid alkyl esters, and at least one member of the group consisting of glycerol, carbohydrate, and mixtures thereof.

The above method, wherein said culture medium comprises (or consists essentially of or consists of) biodiesel co-product stream.

The above method, wherein said culture medium comprises (or consists essentially of or consists of) the product of the transesterification of triacylglycerols or said culture medium is the product of the hydrolysis of triacylglycerols into free fatty acids and the esterification of said free fatty acids.

The above method, wherein (1) a component of said culture medium is a product of the transesterification of triacylglycerols, (2) said culture medium component is a product of the hydrolysis of triacylglycerols into free fatty acids and the subsequent esterification of said free fatty acids, or (3) said culture medium component is the product of the esterification of the free fatty acids and the transesterification of the acylglycerols in a material.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

TABLE 1
The Structural Forms of Sophorolipids Produced by Candida bombicola
from Glycerol, the Biodiesel Coproduct Stream (BCS), and FAME^{a}

| | Fatty Acid Structural Form (mol %) | | |
|---|---|---|---|
| Substrate | FFA^{a} | FAME^{a} | Lactone^{a} |
| Glycerol | 1 | — | 99 |
| | | | (C16:0, 23%)^{b} |
| | | | (C18:0, 33%) |
| | | | (C18:1, 34%) |
| BCS | 32 | 43 | 25 |
| | (C18:1, 16%) | (C18:1, 20%) | (C18:1, 11%) |
| | (C18:2, 15%) | (C18:2, 17%) | |
| Glycerol + FAME^{a} | 24 | 45 | 31 |
| | (C18:1, 13%) | (C18:1, 20%) | (C18:0, 10%) |
| | (C18:2, 10%) | (C18:2, 18%) | (C18:1, 12%) |

^{a}Abbreviations: FFA = Free Fatty Acid; FAME = Fatty Acid Methyl Ester; Lactone = 1′,4″ lactonization of the fatty acid.
^{b}Parenthetical values correspond to specific fatty acids that are in the majority in the designated structural form and the concentrations (in mol %) of those fatty acids.

TABLE 2
Sophorolipid^{a} open-chain content and distribution (in mol %) from
methyl, ethyl and propyl-soyates.

| | | Distribution | | | | | | | | | | |
|---|---|---|---|---|---|---|---|---|---|---|---|---|
| | | Free Acid | | | | | | | Alkyl Ester | | | |
| Carbon | | C_{18:0} | C_{18:1} | | C_{18:2} | | Glycerol Ester^{c} | | C_{18:0} | C_{18:1} | C_{18:2} | |
| Source^{b} | Total Content | ω-1 | ω-1 | ω | ω-1 | ω | C_{18:1} | C_{18:2} | ω-1 | ω-1 | ω-1 | ω |
| Me-Soy | 71 | 2 | 8 | 3 | 7 | 3 | 2 | 4 | 5 | 18 | 15 | 4 |
| Et-Soy | 43 | 4 | 11 | 3 | 17 | 5 | 1 | 2 | 0 | 0 | 0 | 0 |
| Pro-Soy | 80 | 7 | 23 | 5 | 21 | 10 | 5 | 9 | 0 | 0 | 0 | 0 |

^{a}Percentages were derived from the TIC of LC/APCI-MS.
^{b}Carbon sources correspond to the fatty acid esters derived from transesterification of soy oil with methanol (Me-Soy), ethanol (Et-Soy) or propanol (Pro-Soy).
^{c}For structures of glycerol esters see Nunez, A., et al., Chromatographia, 53: 673-677 (2001).

TABLE 3
Sophorolipid^{a} lactone content and distribution (in mol %) from
methyl, ethyl and propyl soyates.

| | | Distribution | | | | | | |
|---|---|---|---|---|---|---|---|---|
| Carbon | | C_{16:0} | | C_{18:0} | | C_{18:1} | | C_{18:2} |
| Source^{b} | Total Content | ω-1 | ω | ω-1 | ω | ω-1 | ω | ω-1 + ω |
| Me-Soy | 28 | 1 | 1 | 6 | Tr^{c} | 15 | 1 | 4 |
| Et-Soy | 57 | 3 | 4 | 18 | Tr^{c} | 20 | Tr^{c} | 12 |
| Pro-Soy | 20 | 1 | 1 | 9 | Tr^{c} | 6 | Tr^{c} | 3 |

^{a}Percentages were derived from the TIC of LC/APCI-MS.
^{b}For explanation of abbreviations see footnotes from Table 2.
^{c}Tr is less than 0.5 mol %.

We claim:

1. A method for the production of open-chained sophorolipids, said method comprising cultivating yeast capable of producing open-chained sophorolipids in a culture medium comprising fatty acid alkyl esters and at least one member of the group consisting of glycerol, carbohydrate, and mixtures thereof; wherein at least about 60% of the sophorolipids produced are open-chained sophorolipids.

2. The method according to claim 1, wherein said yeast is selected from the group consisting of Candida bombicola, Candida apicola, and mixtures thereof.

3. The method according to claim 1, wherein said yeast is Candida bombicola.

4. The method according to claim 1, wherein said yeast is Candida bombicola NRRL Y-30816.

5. The method according to claim 1, wherein at least about 65% of the sophorolipids produced are open-chained sophorolipids.

6. The method according to claim 1, wherein at least about 70% of the sophorolipids produced are open-chained sophorolipids.

7. The method according to claim 1, wherein at least about 75% of the sophorolipids produced are open-chained sophorolipids.

8. The method according to claim 1, wherein at least about 80% of the sophorolipids produced are open-chained sophorolipids.

9. The method according to claim 1, wherein at least about 85% of the sophorolipids produced are open-chained sophorolipids.

10. The method according to claim 1, wherein said fatty acid alkyl esters are fatty acid methyl esters, ethyl esters, propyl esters, or mixtures thereof.

11. The method according to claim 1, wherein said culture medium contains glycerol.

12. The method according to claim 1, wherein said culture medium contains carbohydrate.

13. The method according to claim 1, wherein said culture medium contains glycerol but not carbohydrate.

14. The method according to claim 1, wherein said culture medium consists essentially of fatty acid alkyl esters, carbohydrate, and optionally free fatty acids.

15. The method according to claim 1, wherein said culture medium consists essentially of fatty acid alkyl esters, glycerol, and optionally free fatty acids.

16. The method according to claim 1, wherein said culture medium does not contain free fatty acids.

17. The method according to claim 1, wherein said culture medium consists essentially of fatty acid alkyl esters, and at least one member of the group consisting of glycerol, carbohydrate, and mixtures thereof.

18. The method according to claim 1, wherein said culture medium comprises biodiesel co-product stream.

19. The method according to claim 1, wherein said culture medium comprises the product of the transesterification of triacylglycerols or said culture medium is the product of the hydrolysis of triacylglycerols into free fatty acids and the esterification of said free fatty acids.

20. The method according to claim 1, wherein said method does not involve a lactone ring-opening step to produce said open-chained sophorolipids.
